(19) United States
(12) Patent Application Publication
(10) Pub. No.: US 2016/0022544 A1
(43) Pub. Date: Jan. 28, 2016

(54) GASTROSTOMY DELIVERY SYSTEM AND METHOD

(71) Applicant: R. Scott TURNER, Palmerton, PA (US)

(72) Inventor: R. Scott TURNER, Palmerton, PA (US)

(21) Appl. No.: 14/444,387

(22) Filed: Jul. 28, 2014

(51) Int. Cl. A61J 15/00; A61M 39/24; A61M 39/10

(52) U.S. Cl. CPC A61J 15/0015; A61M 39/1011; A61M 39/24; A61J 15/0026

(57) ABSTRACT

A method of feeding a patient through the patient's gastrostomy feeding tube. The method includes the steps of connecting a connector of a filled feeding bag directly to a port of the feeding tube; manipulating the feeding bag such that the fluid within the filled feeding bag is driven through the connector and into the feeding tube; and disconnecting the feeding bag connector from the port of the feeding tube after the fluid has been expelled from the feeding bag.

### FIELD OF THE INVENTION

This invention relates to gastrostomy fluid delivery. More particularly, the invention relates to a system and method for delivery of fluids through a gastrostomy feeding tube.

### BACKGROUND OF THE INVENTION

Percutaneous endoscopic gastrostomy (PEG) is an endoscopic medical procedure in which a tube (PEG tube) is passed into a patient's stomach through the abdominal wall, most commonly to provide a means of feeding when oral intake is not adequate. This provides enteral nutrition (making use of the natural digestion process of the gastrointestinal tract) despite bypassing the mouth. In addition to the PEG procedure, a gastrostomy feeding tube may be placed using an open surgical gastrostomy insertion procedure. While the description herein refers to a PEG feeding tube, it is recognized that the present invention may be utilized with feeding tubes regardless of the procedure utilized to position the tube with respect to a patient.

Referring to FIG. 1, the PEG tube 10 typically extends from a site 12 on the patient's abdomen 14. The opposite end of the tube 10 includes a connection fitting 16 or the like with one or more ports 17, 19 for connection of a supply apparatus. A clamp 18 may be positioned along the tube 10 to seal the tube 10. Once the PEG tube is installed, the patient may be fed, provided medicine or the like through one of the ports 17, 19. Currently the process of PEG feeding is very tedious, prone to fluid spills, slow and awkward to administer, and prone to introduce infection.

The most prevalent method of providing nutrition fluid to a patient requiring PEG feeding is illustrated in FIG. 1. The feeding fluid typically comes in 8 ounce cans 30 and the fluid from these cans must be poured into a syringe 22 for bolus feeding. In many cases, because of the high viscosity of the fluid, it must be forced out of syringe 22 using a plunger 24. The person doing the feeding must open the can/package 30 of food and pour it into the (2 ounce) syringe 22 and then plunge the food into the tube 10. For an 8 ounce feeding, this process is commonly repeated a total of four times. After each of these partial feedings, the syringe 22 must be removed from the respective port 17/19 for refilling of the syringe 22. The plunger 24 cannot simply be removed from the syringe 22 since such action would draw the fluid back into the syringe 22. The required steps are to close clamp 18, remove syringe 22 from the port 17/19, pull plunger 24 out of syringe 22, cap the syringe stem, fill the syringe 22 from the can 30, reinsert the plunger 24, invert syringe 22 and remove cap, reconnect the syringe 22 to the port 17/19, open the clamp 18 and push down the plunger 24. Since the can 30 and the syringe 22 are each held in a hand during the process, the administrator typically must set down plunger 24 to fill the syringe 22. After the first round of partial feeding, the plunger 24 will have fluid thereon which will drip onto the surface on which the plunger 24 is set. It should be obvious the risk of a spill, misstep, complication and contamination is likely during this involved repetitive process. Additionally, the time to complete a feeding is extended because of this involved repetitive process.

The complications with the plunger may avoided by instead allowing the food to be delivered by gravity. Such a method has its own drawbacks. For example, since the food is not pushed by a plunger, the feeding can take much longer. During this extended time, the administrator is often forced to hold the syringe 22 in an elevated position.

As an alternative to bolus feeding, feeding may be provided through a mechanical pump 40 illustrated in FIG. 2. A feeding fluid fillable bag 42 is supported by a pole or the like and a feeding line 44 extending therefrom is passed through the pump 40 and then connected to one of the ports 17, 19 of the PEG tube 10. While addressing some of the problems with bolus feeding, pump feeding raises additional problems. First, pump systems are rather cumbersome with its size on a pole with a large footprint base, a power supply wire and a snagable tube to the patient. Furthermore, the bag-tube set on the pump 40 must be changed on a daily basis for sanitary reasons and the pump 40 often must be frequently reset. The pump 40 is typically configured to deliver the fluid in a slow, continuous manner. In principle, pumps need only be used when the administration of fluid flow needs to be very slow and controlled. The need for pump administration most commonly occurs when a patient's digestive system is being reintroduced to nutrition. Cumbersome portable backpack pumps (not shown) are prescribed to mobile people most commonly simply because it is the easiest way to get liquid food into them.

In another method, most commonly used in nursing homes, the nutritional fluid comes in large collapsible bags 1 liter or more and is configured to hang on a pole. A flexible supply tube is molded into the bottom of the bag at one end and has a PEG tube fitting on the other. The fluid flow rate is controlled by pinching the supply tube with a roller clamp. The down side of this method is the bag is relatively large, and as a result, it must be hung above the patient to have the fluid flow. Squeezing this bag requires a lot of force due to its large surface area thus making it impractical for manually expelling the fluid for fast and convenient feeding. Also, because of the large-size, the flow rate must be carefully monitored to prevent over feeding of the patient. Furthermore, the large size is cumbersome and inconvenient for many PEG feeding applications.

In nearly all cases, due to the complications involved in PEG feeding, most commonly in home environments, the caregiver or patient becomes frustrated. As a result, the outcome is often very unsatisfactory.

### SUMMARY OF THE INVENTION

Compared to the prior art delivery methods, the present invention provides a single dose feeding delivery container that attaches directly to a PEG tube fitting and the rate of fluid delivery can be easily controlled by driving or otherwise delivering fluid from the feeding bag. It has been demonstrated that the fluid can be delivered in as little as one minute. As a result, it should be obvious that a method that only involves opening a fluid container, connecting it to port, opening a clamp, delivering the fluid, closing clamp and disconnecting the feeding bag is a far easier, faster and safer method of delivering fluid to a person. In some embodiments wherein the PEG tube fitting includes a check valve, the steps of opening and closing the clamp may be removed.

In at least one embodiment, the present invention provides a method of feeding the patient through the patient's gastrostomy feeding tube. The method includes releasing a seal preventing a fluid within the filled feeding bag from exiting; connecting the filled feeding bag directly to a port of the feeding tube; opening a clamp on the feeding tube, driving the fluid within the filled feeding bag such that it is discharged through the connector and into the feeding tube; closing the clamp and disconnecting the feeding bag connector from the port of the feeding tube.

In at least one embodiment of the invention, the feeding bag is a pre-filled, sealed bag.

In at least one embodiment of the invention, the feeding bag is refillable and re-sealable.

In at least one embodiment of the invention, the feeding bag connector is integrally formed with the feeding bag.

In at least one embodiment of the invention, the filled feeding bag has an empty overall approximate internal width of 2¾ inches.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a perspective view illustrating a syringe bolus feeding procedure.

FIG. 2 is a perspective view illustrating a pump feeding procedure.

FIG. 3 is a plan view of an exemplary filled feeding bag in accordance with an embodiment of the invention.

FIGS. 4 and 5 are partial perspective views of another exemplary filled feeding bag in accordance with an embodiment of the invention in sealed and unsealed conditions, respectively.

FIG. 6 is a cross-sectional view of another exemplary filled feeding bag in accordance with an embodiment of the invention.

FIGS. 7A and 7B are exploded and assembled perspective views, respectively, of an alternative connection fitting including a seal puncture spear and a check valve.

FIG. 8 is a perspective view of yet another exemplary filled feeding bag in accordance with an embodiment of the invention wherein the bag includes a bag filling port.

FIG. 9 is a flow diagram illustrating an exemplary method of feeding a patient through the patient's gastrostomy feeding tube.

FIG. 10 is a perspective view illustrating the filled feeding bag of FIG. 1 prior to connection to a PEG feeding tube.

FIG. 11 is a perspective view similar to FIG. 10 illustrating the filled feeding bag of FIG. 1 connected to the PEG feeding tube.

FIGS. 12 and 13 are perspective views similar to FIG. 10 illustrating progression of an exemplary method of expelling of fluid from the filled feeding bag of FIG. 1 into the PEG feeding tube.

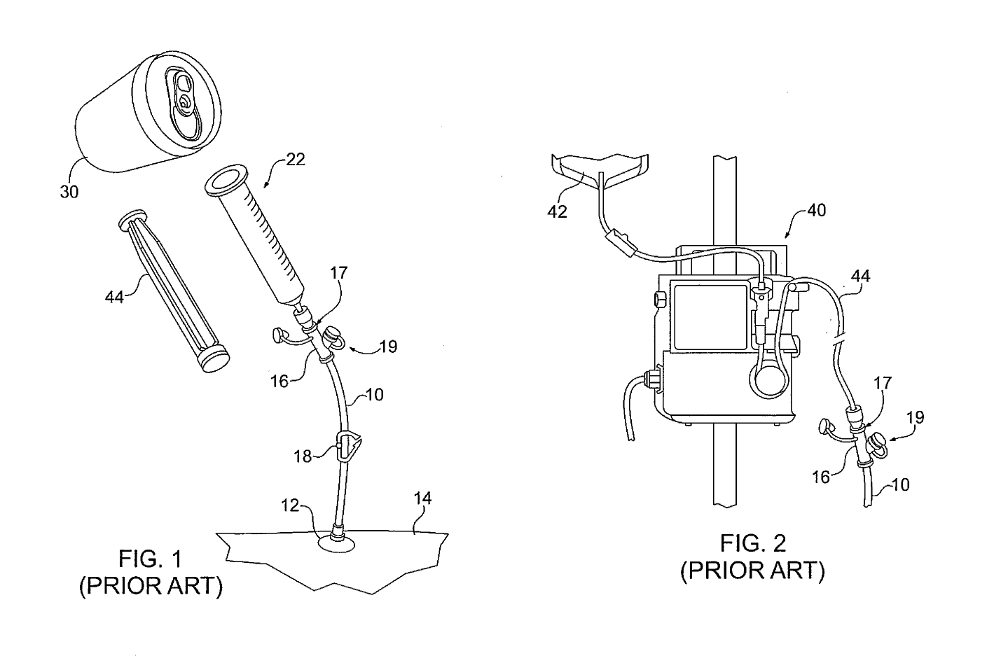

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

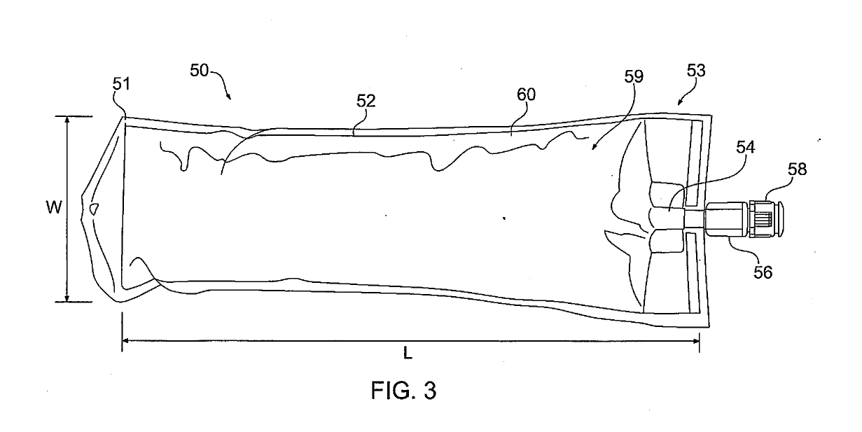

Referring to FIG. 3, an exemplary filled feeding bag 50 in accordance with an embodiment of the invention will be described. The filled feeding bag 50 includes a tubular body 52 extending from a closed end 51 to a connection end 53 with an internal chamber 59 defined therebetween. The internal chamber 59 preferably has a volume equal to or slightly larger than the amount of fluid 60 intended to be delivered. For example, the internal chamber 59 may have a volume of 8 fluid ounces to correspond to the volume of a typical feeding can 30. The tubular body 52 is preferably manufactured from a flexible material such that the tubular body 52 may be compressed to expel fluid from within the chamber 59. To assist with expelling of the fluid, the tubular body 52 preferably has a narrow configuration, i.e. a length L larger than the width W. An exemplary tubular body 52 may be approximately 7½ inches long by 2¾ inches wide. In a preferred embodiment, the tubular body has a length L to width W ratio of at least 2 to 1, and more preferably a ratio of at least 2.75 to 1, however, it is recognized that the width W may be maintained while the length L is adjusted to accommodate different volumes of fluid. With this configuration, when the tubular body 52 is squeezed, rolled or otherwise compressed, the fluid 60 is driven toward the connection end 53.

To further facilitate driving of the fluid from the chamber 59, the illustrated tubular body 52 tapers to a neck 54 which in turn is connected to a connector 56. The connector 56 is configured to connect with a port of a PEG feeding tube 10. Pursuant to recent regulations, the connector 56 preferably has the form of a luer connector, however, it may have other configurations. In the current embodiment, a cap 58 is positioned over the luer connector 56 until the feeding bag 50 is ready for use. The cap 58 maintains the sterility of the connector 56 and also maintains the fluid 60 or other fluid remains sealingly within the chamber 59 until the cap 58 is removed.

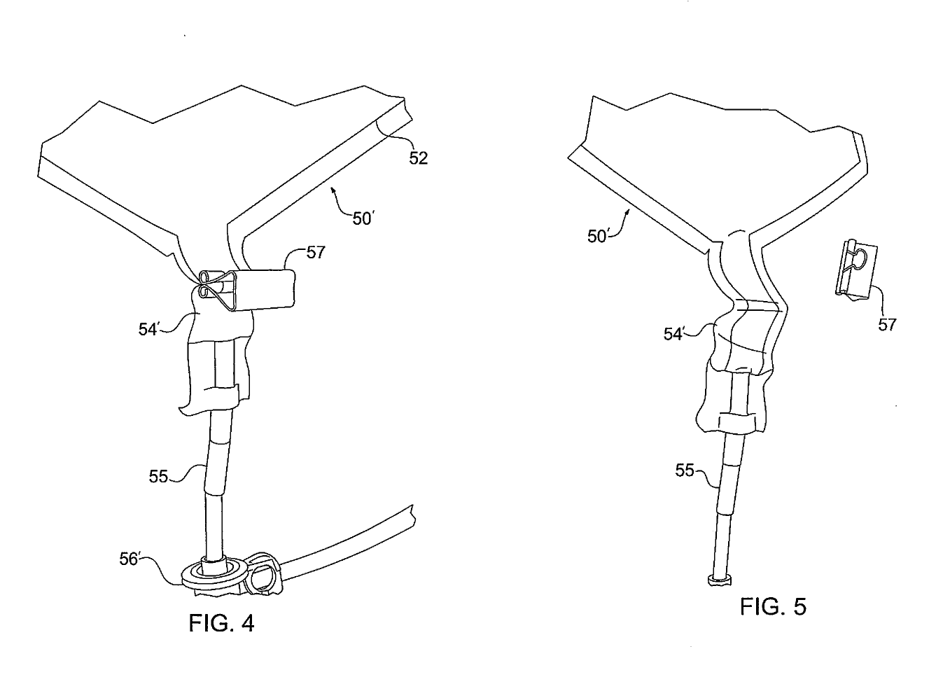

Additionally, the seal may take other configurations other than a cap. Referring to FIGS. 4 and 5, another exemplary filled feeding bag 50′ is illustrated. The feeding bag 50′ is substantially the same as the previous embodiment except that the bag 50′ includes an elongated neck 54′ with a clamp 57 positioned thereon to seal the bag 50′. The clamp 57 is opened and removed from the neck 54′ when it is desired to unseal the bag 50′. A tube 55 may extend from the elongated neck 54′ to the connector 56′.

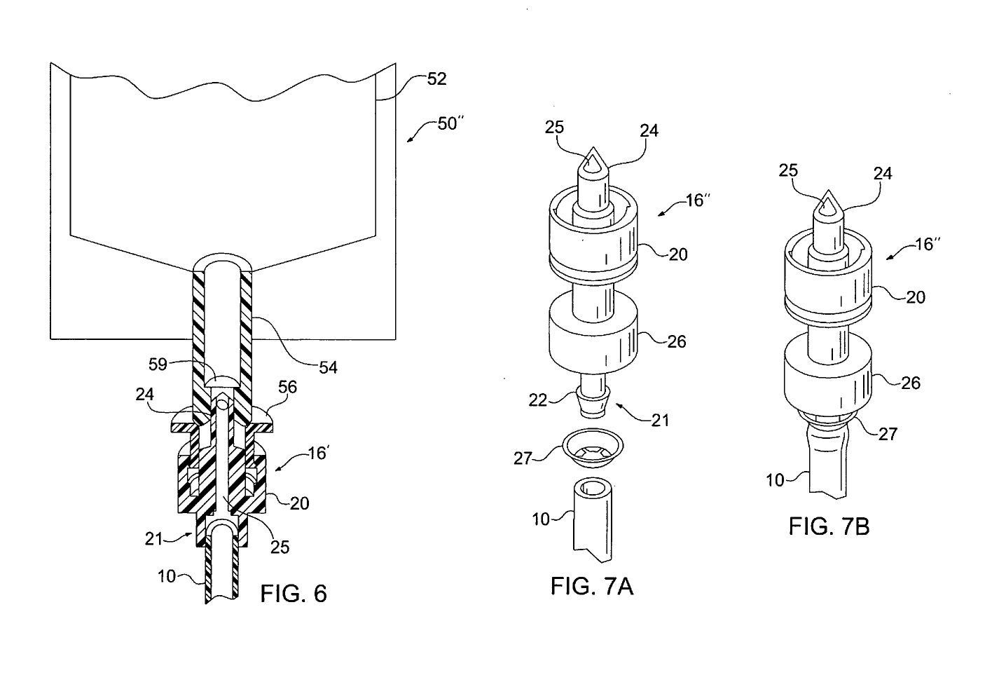

Referring to FIG. 6, another exemplary filled feeding bag 50″ is illustrated. The feeding bag 50″ is substantially the same as the first embodiment, except that the bag 50″ includes a sealing membrane 59 extending across the neck 54. The membrane 59 may be configured to be punctured as the connector 56 is connected to the connection fitting 16′ of the PEG tube 10. In such an embodiment, the connection fitting 16′ includes a luer connector 20 with a spike 24 extending therefrom. When the connection fitting 16′ is fully connected to the connector 56, the spike 24 punctures the membrane 59. A lumen 25 extends through the connection fitting 16′ such that fluid within the bag 50″ will flow to the PEG tube 10, connected at the opposite end 21 of the connection fitting 16′, once the spike 24 punctures the membrane 59.

While puncturing of the membrane seal 59 is illustrated, the membrane 59 may be configured such that sufficient pressure within the bag 50, for example, by squeezing the tubular body 52, may cause the membrane 59 to break. As another alternative, the membrane 59 may be manufactured from a frangible material such that squeezing or bending of the neck 54 will cause the membrane 59 to break. Such examples are provided for illustrative purposes only and various mechanisms and methods may be utilized to break the membrane seal 59. Additionally, sealing of the filled feeding bag 50, 50′, 50″ is not limited to the illustrated cap 58, clamp 57 or membrane 59 and it is understood that other mechanisms and methods may be utilized.

Referring to FIGS. 7A and 7B, an alternative connection fitting 16″ will be described. The alternative connection fitting 16″ includes an optional check valve 26 positioned along the lumen 25 to prevent backflow from the PEG tube 10. While not illustrated, it is noted that a control port may be positioned between the check valve 26 and the PEG tube 10 to facilitate controlled back flow if desired. Additionally, a barb 22 is provided on end 21 to facilitate a more positive connection to the PEG tube 10. The barb 22 is configured such that a pulling force away from the fitting 16″ causes the barb 22 to bite into and retain the PEG tube 10. At times it may be desirable to change the fitting 16″, or remove the fitting 16″ for additional cleaning, without disconnecting the PEG tube 10 from the patient. In the illustrated embodiment, a tapered ring 27 is positioned between the check valve 26 and the PEG tube 10 to assist with disconnection of the PEG tube 10. Pushing of the tapered ring 27 away from the fitting 16″ will push the PEG tube 10 over the barb 22 without the barb 22 biting into the tube. The PEG tube 10 can then be connected over the barb 22 of a cleaned or new fitting 16″.

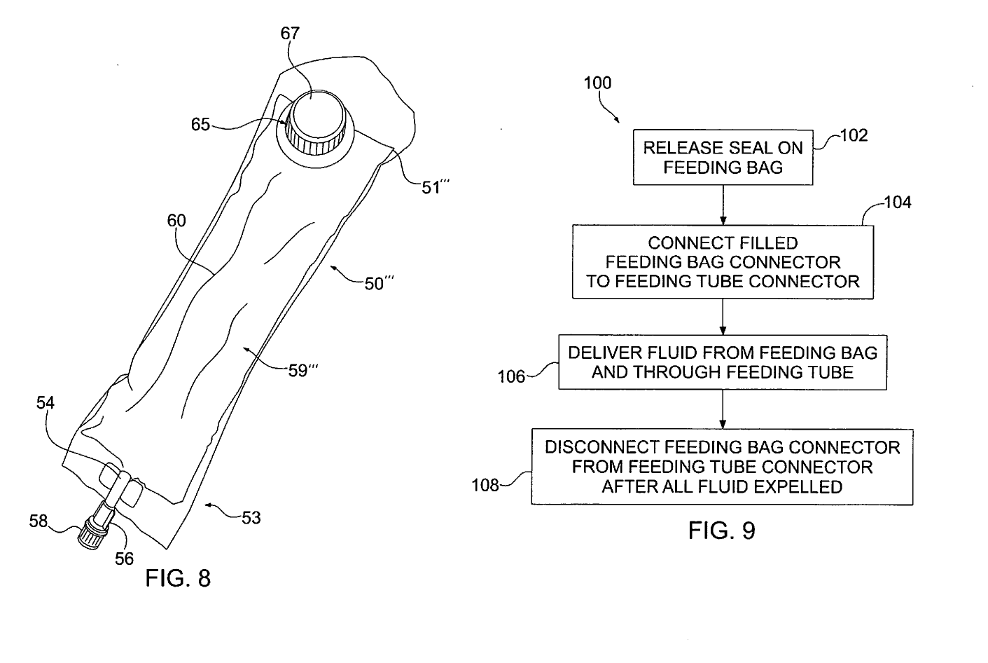

In the embodiments illustrated in FIGS. 3-6, the chambers 59 are prefilled with a desired amount of fluid 60 and the closed end 51 is factory sealed. Such bags 50, 50′, 50″ are intended to be disposable. FIG. 8 illustrates another exemplary filled feeding bag 50′″ which is configured to be reusable. The feeding bag 50′″ is substantially the same as the previous embodiments except that the closed end 51′″ includes an opening 65 configured to be closed by a resealable closure mechanism 67, for example, a threaded cap. The invention is not limited to such a closure and may take other forms, for example, a plastic zipper or a folded over section retained with a clamp or the like. With such a configuration, a user may fill the internal chamber 59′″ with a desired amount of fluid 60 and then seal the closure mechanism 67 and the filled bag 50′″ is ready for use in a manner similar to the prefilled bags 50, 50′, 50″.

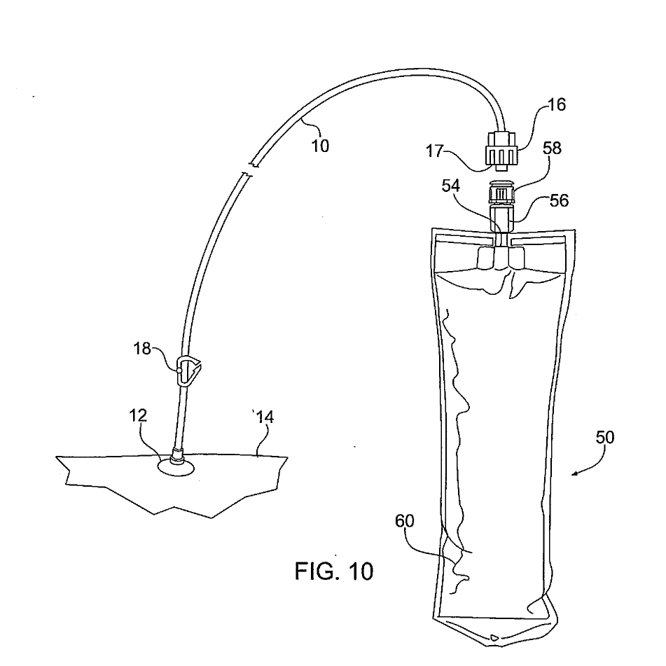

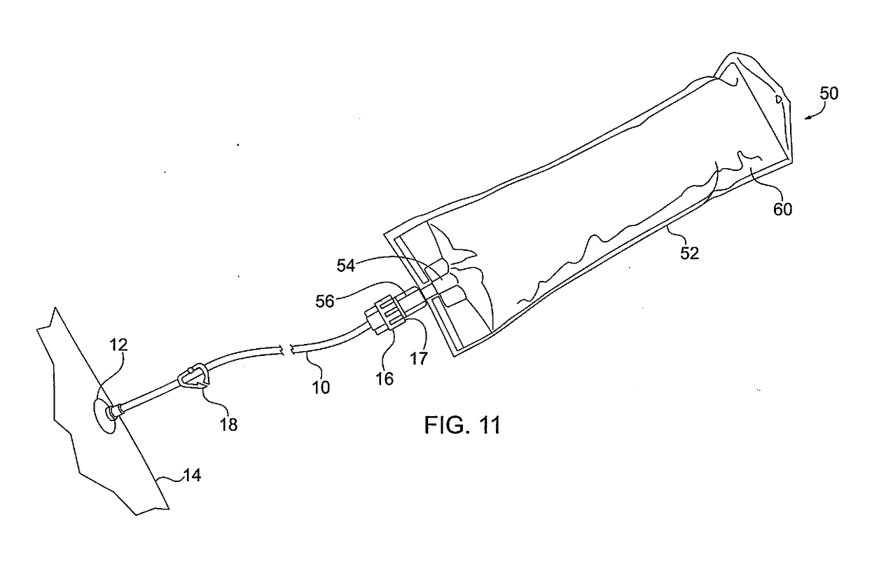

Having described exemplary filled feeding bags 50, 50′, 50″, 50′″, an exemplary method 100 of feeding a patient utilizing one of the bags 50, 50′, 50″, 50′″ will be described with reference to FIGS. 9-13. For the bag 50, in a first step 102, the seal is released from the bag 50 by removing the cap 58. Thereafter, in step 104, the connector 56 of the filled bag 50 is connected within one of the ports 17 of the feeding tube connection fitting 16, as illustrated in FIGS. 10 and 11. In the other embodiments, these steps may be reversed or combined. For example, for the bag 50′ illustrated in FIGS. 4 and 5, the connector 56 may be connected first and then the seal released by removing the clamp 57. For the bag 50″ illustrated in FIGS. 6 and 7, the steps may be combined as the spike 24 will puncture the membrane 59 to release the seal as the connector 56 is connected. With the connector 56 connected and the seal released, the fluid 60 may flow from the internal chamber 59 into the feeding tube 10 and into the patient.

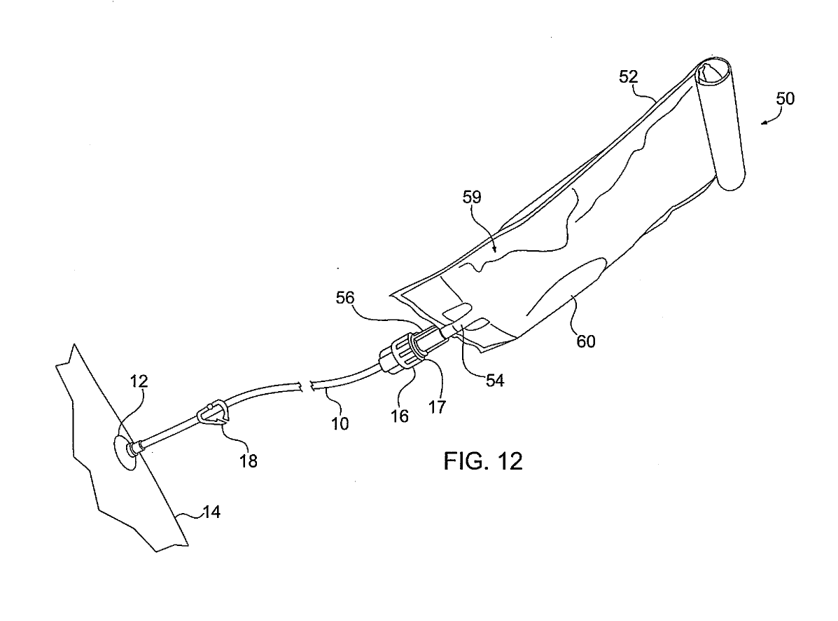

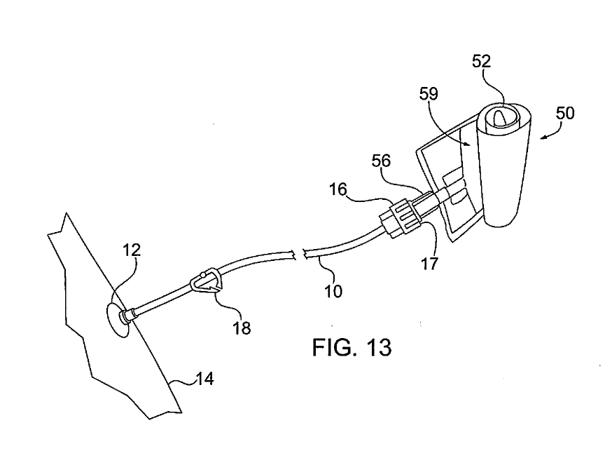

The bag 50 may either be raised up, manually squeezed, pressed, rolled as illustrated in FIGS. 12 and 13, or otherwise manipulated to drive the fluid from the bag 50 to flow into the patient. This process can take as little as 1 minute by squeezing the bag, or as long as the patient wants by raising the bag for slow gravity feed. The illustrated method includes the step 106 of driving the fluid from the feeding bag 50 and through the feeding tube. The driving of the fluid may result from a user force or gravity. In step 108, once the fluid 60 has been expelled from the chamber 59, the connector 56 is disconnected from the connection fitting 16. If the bag 50 is disposable, it can thereafter be disposed of, however, if it is a reusable bag 50′″, the bag 50′″ may be cleaned and refilled for future use.

The benefits of a pre-filled collapsible bag are many. The nutritional fluid is never poured, thus eliminating spillage and contact with foreign materials that can cause contamination. This reduces the potential for bacteria which may cause GI distress. The system and method also prevent leakage through disconnection or spillage from pouring fluid from a can into a syringe. The system and method also saves time in feeding, since fluid can be squeezed through the tube 10. Additionally, the bags are very portable and when empty are small and easily disposed of.

With refillable collapsible feeding bags, users have the option to interchange with traditional fluids, introduce medication, use tap, distilled or bottled water. The bags can have wide mouth openings that can be easily sealed and closed. The caregiver or patient can administer the fluid as quickly as a minute if desired.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. A method of feeding a patient through the patient's gastrostomy feeding tube, the method comprising the steps of:
connecting a connector of a filled feeding bag directly to a port of the feeding tube;
manipulating the feeding bag such that a fluid within the filled feeding bag is driven through the connector and into the feeding tube; and
disconnecting the feeding bag connector from the port of the feeding tube after the fluid has been driven from the feeding bag.

2. The method of claim 1 further comprising the step of releasing a seal between a fluid within the filled feeding bag and the connector.

3. The method of claim 2 wherein the step of releasing the seal is performed before the connecting step.

4. The method of claim 3 wherein the seal is provided in part by a cap on the connector.

5. The method of claim 2 wherein the step of releasing the seal is performed after the connecting step.

6. The method of claim 5 wherein the seal is provided in part by a clamp positioned on an extended neck of the feeding bag.

7. The method of claim 2 wherein the step of releasing the seal is performed simultaneously with the connecting step.

8. The method of claim 7 wherein the seal is provided in part by a membrane within a neck of the filled bag.

9. The method of claim 8 wherein a connector on the port of the feeding tube includes a spike which punctures the membrane as the connector on the feeding bag is connected with the connector on the port.

10. The method of claim 1 wherein the filled feeding bag is a pre-filled, sealed bag.

11. The method of claim 1 wherein the filled feeding bag is a filled resealable bag which is filled and sealed prior to the connecting step.

12. The method of claim 1 wherein the filled feeding bag connector is integrally formed with the feeding bag.

13. The method of claim 1 wherein the filled feeding bag has a length to width ratio of at least 2 to 1.

14. The method of claim 1 wherein the filled feeding bag has a volume of fluid equivalent to the volume of a single feeding.

15. The method of claim 1 wherein the manipulating step includes squeezing of the feeding bag.

16. The method of claim 1 wherein the manipulating step includes rolling of the feeding bag.

17. The method of claim 1 wherein the manipulating step includes raising the feeding bag such that gravity drives the fluid from the bag.

18. A connection fitting for connection to a PEG feeding tube, the connection fitting comprising:
a connector at a first end and barb at a second end thereof with a passageway extending between the first and second ends, wherein the barb is configured to be received within the PEG feeding tube and to bite into the tube when the tube is pulled away from the connection fitting; and
a tapered ring positioned between the first and second ends with a narrower end of the ring facing toward the second end; wherein the narrower end is configured to engage the PEG feeding tube and push the PEG feeding tube up to the barb to disconnect the connection fitting from the PEG feeding tube.

19. The connection fitting of claim 18 wherein the connector is a luer connector.

20. The connection fitting of claim 18 wherein a check valve is positioned between the first and second ends and extends across the passageway.
